(19) United States
(12) Patent Application Publication
(10) Pub. No.: US 2019/0047856 A1
(43) Pub. Date: Feb. 14, 2019

(54) PHOTO-THERMAL REACTIONS OF ALCOHOLS TO HYDROGEN AND ORGANIC PRODUCTS OVER METAL OXIDE PHOTO-THERMAL CATALYSTS

(71) Applicant: SABIC Global Technologies B.V., Bergen op Zoom (NL)

(72) Inventors: Muhammad Amtiaz NADEEM, Thuwal (SA); Hicham IDRISS, Thuwal (SA)

(21) Appl. No.: 16/077,773

(22) PCT Filed: Apr. 20, 2017
(86) PCT No.: PCT/IB2017/052285

§ 371 Date: Aug. 14, 2018

Related U.S. Application Data
(60) Provisional application No. 62/332,592, filed on May 6, 2016.

(51) Int. Cl. C01B 3/22; C07C 45/00; B01J 21/06; B01J 23/52; B01J 35/00

(52) U.S. Cl. CPC C01B 3/22; C07C 45/002; B01J 21/063; C01B 2203/1229; B01J 35/004; C01B 2203/0277; B01J 23/52

(57) ABSTRACT

Photo-thermal catalysts and methods of use are described. The photo-thermal catalyst can include a photo-active metal oxide and, optionally, a plasmon resonance material. The photo-thermal catalyst has a temperature of 150° C. to 400° C. and is in contact with electromagnetic radiation. The photo-thermal catalyst can be used in a photo-thermal method to generate hydrogen from alcohols.

### CROSS REFERENCE TO RELATED APPLICATIONS

This application claims benefit to U.S. Provisional Application No. 62/332,592 to Nadeem et al., filed May 6, 2016, which is incorporated in its entirety herein without disclaimer.

### BACKGROUND OF THE INVENTION

A. Field of the Invention

The invention generally concerns the production of hydrogen gas (H₂) and an organic product from an alcohol. In particular, the invention relates to irradiating a thermally-heated metal oxide photocatalyst in the presence of an alcohol to produce H₂ and an organic product.

B. Description of Related Art

A large number of photocatalytic materials including oxides, nitrides, and sulfides have been studied for hydrogen production with the aim to develop a viable system for industrial applications. Additionally, different catalyst modifications such as noble metal loading, addition of sacrificial reagent dye sensitization, anion doping, and metal ion-implantation have been evaluated. However, all of these strategies have, so far, failed to enhance the hydrogen production rates acceptable for industrial applications.

Various attempts have been made to produce hydrogen through the photocatalytic or photo-electrical water-splitting reactions. Photoreaction rates on TiO₂ based catalysts, however, have been low, thereby making photocatalytic applications on industrial scale economical unfeasible. By way of example, photocatalytic water splitting using sacrificial agents, has only been able to deliver hydrogen production rates at a 5% solar photon to hydrogen conversion (STH).

Due to the low production of hydrogen from water, various attempts to generate hydrogen from sources other than water have been investigated. Alcohols such as ethanol have been investigated as an alternate source to water in photocatalytic reactions to produce hydrogen. By way of example, Murdoch, et al., in Nat Chem, 2011, 3(6), p. 489-492 describes photocatalytic hydrogen production from ethanol over Au/TiO₂ nanoparticles. However, these photocatalysts suffer in that they can have high electron-hole recombination rates, thereby reducing the efficiency of the reaction. In another example, Kolinko et al. (Theoretical and Experimental Chemistry, 2015, 51(2), p. 96-103) describes photocatalytic oxidation of ethanol vapor to acetaldehyde using TiO₂ modified with noble metal using visible light. This process also suffers from low rates of acetaldehyde and hydrogen formation.

Aldehydes have also been investigated as alternative sources to water for the production of hydrogen. By way of example, Falconer et al. (Journal of Catalysis, 1998. 179(1): p. 171-178) describes photocatalytic and thermal catalytic oxidation of low concentrations of acetaldehyde on Pt/TiO₂, and Wu et al. (The Journal of Physical Chemistry B, 2006. 110(19): pp. 9627-9631) describes formation of crotonaldehyde from the decomposition of ICH₂CH₂OH on powdered TiO₂. These processes suffer from catalyst deactivation due to by-products on the TiO₂ surface.

While various attempts to produce hydrogen from sources other than water have been investigated, these attempts suffer from operational inefficiencies, catalyst deactivation, and high electron-hole recombination rates.

### SUMMARY OF THE INVENTION

A solution to the problems associated with the photocatalytic production of hydrogen from organic compounds such as alcohols has been discovered. The solution is premised on irradiating a thermally heated photocatalyst having alcohol adsorbed on its surface to produce hydrogen and organic products such as aldehydes or ketones. The thermally heated photo catalyst can include a photo-active metal oxide and optionally a plasmon resonance active metal dispersed on the surface of the metal oxide. When the photo-thermal catalyst is thermally heated to temperature of 150° C. to 400° C. (e.g., bulk as well as a surface temperature of the catalyst) and irradiated with electromagnetic radiation, alcohol (e.g., C₁₋₃ alcohols such as methanol, ethanol, and propanol) adsorbed on the surface of the catalyst can be converted to hydrogen and an organic product (e.g., an aldehyde and/or ketone). Without wishing to be bound by theory, it is believed that thermally heating the surface of the catalyst (e.g., localized heating) prior to or during irradiation facilitates interaction between the photo-active material and the alcohol adsorbed on the catalyst surface. Localized heating can help electrons cross the activation barrier faster and/or shift in the reaction mechanism due to stabilization/destabilization of the reaction intermediates. This results in increased hydrogen and an organic product production when compared with the same catalyst that has not been thermally heated (e.g., temperature of catalyst is room temperature or 15° C. to 30° C.). By way of example, and as shown in non-limiting embodiments in the Examples section, an increase of acetaldehyde (dehydrogenation of the alcohol) production by three orders of magnitude and an increase in hydrogen production by one order of magnitude can be obtained with a thermally heated catalyst (e.g., at 175° C. and 350° C.) as compared to the same catalyst that was not thermally heated. It is also believed that adsorption of the alcohol on the metal oxide promotes dehydrogenation of the alcohol with less by-product formation.

In a particular aspect of the present invention, a photo-thermal method for producing hydrogen (H₂) and an organic product from an alcohol is described. The method can include irradiating a thermally heated photo-thermal catalyst of the present invention that has alcohol adsorbed on its surface with electromagnetic radiation to produce H₂ and the organic product from the alcohol. The thermally-heated photo-thermal catalyst can have a temperature of 150° C. to 400° C. (e.g., 250° C. to 400° C., preferably 300° C. to 400° C., or more preferably 325° C. to 375° C., or most preferably about 355° C.). The temperature can refer to the surface temperature of the catalyst. The alcohol can be a C₁₋₃ alcohol (e.g., methanol, ethanol, propanol, or any combination thereof) and the organic compound can be formaldehyde, acetaldehyde, propyl aldehyde or acetone. The photo-thermal catalyst can include titanium dioxide (TiO₂), cerium dioxide (CeO₂), zinc oxide (ZnO), or vanadium oxide (V₂O₅) or any combination thereof. In some preferred instances, (TiO₂) can be used. In some embodiments, the photo-thermal catalyst includes one or more plasmon resonance active metals (e.g., silver (Ag), gold (Au), or copper (Cu)) or alloys thereof. In a preferred aspect, the photo-thermal catalyst is Ag/TiO₂. The photo-thermal catalyst can include 0.1 to 10 wt. % or 0.3 to 5 wt. % or 0.5 to 3 wt. % of the plasmon resonance active metal based on the total weight of the catalyst. In some instances, the thermally heated photo-thermal catalyst is subjected to an alcohol feed stream to adsorb the alcohol prior to the irradiation step. In another instance, the thermally-heated photo-thermal catalyst can be subjected to an alcohol feed stream during the irradiation step. The electromagnetic radiation can be at a wavelength of 100 nm to 500 nm, preferably 300 nm to 450 nm and can include ultraviolet radiation or sunlight. It was surprisingly found, as shown in one non-limiting Example, that the production of aldehyde decreases in the absence of irradiation.

In yet another aspect of the present invention, photo-thermal catalysts are described. The photo-thermal catalyst can include a photo-active metal oxide and alcohol adsorbed on the surface of the catalyst. The photo-thermal catalyst can have a temperature of 150° C. to 400° C. (e.g., 250° C. to 400° C., preferably 300° C. to 400° C., or more preferably 325° C. to 375° C., or most preferably about 355° C.) and can be in contact with electromagnetic radiation (e.g., a wavelength of 100 nm to 500 nm, preferably 300 nm to 450 nm). The adsorbed alcohol can be a C₁₋₃ alcohol (e.g., methanol, ethanol, n-propanol) and the photo-thermal catalyst is capable of dehydrogenating the alcohol to produce hydrogen and an organic product (e.g., formaldehyde, acetaldehyde, propyl aldehyde, acetone). The photo-thermal catalyst can include a plasmon resonance active metal (e.g., Ag, Au, Cu, or any combination or alloy thereof) dispersed on the photo-active metal oxide (e.g., TiO₂, CeO₂, ZnO, or V₂O₅ or any combination thereof). In a preferred aspect of the present invention, the photo-thermal catalyst can be Ag/TiO₂ having 0.1 to 10 wt. % or 0.3 to 5 wt. % or 0.5 to 3 wt. % of Ag, based on the total weight of the catalyst.

The following includes definitions of various terms and phrases used throughout this specification.

The term “C₁₋₃ alcohol” refers to a hydrocarbon alcohol having 1 to 3 carbon atoms. Non-limiting examples, of C₁₋₃ alcohols include CH₃OH (methanol), CH₃CH₂OH (ethanol), C₃H₅OH (propanol). Examples of propanol include CH₃CH₂CH₂OH (n-propanol), CH₃CHOHCH₃ (isopropanol).

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %,” “mol. %,” or “vol. %” refers to a weight, molar, or volume percentage of a component, respectively, based on the total weight or the total volume of material that includes the component. In a non-limiting example, 10 grams of a component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods and catalysts of the present invention can “comprise,” “consist essentially of” or “consist of” particular ingredients, components, compositions, etc. disclosed throughout the specification. With respect to the transitional phase “consisting essentially of,” in one non-limiting aspect, a basic and novel characteristic of the method and/or catalysts of the present invention are their abilities to produce hydrogen and organic products (aldehydes or ketones) from alcohols.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 is an illustration of a system to produce hydrogen from alcohols using the photo-thermal catalyst of the present invention.

FIG. 2 is an illustration of a proposed reaction mechanism for the formation of hydrogen and acetaldehyde photo-thermal catalyst of the present invention

FIG. 3 is an illustration of another proposed reaction mechanism for the formation of hydrogen and acetaldehyde using the photo-thermal catalyst of the present invention.

FIGS. 4A and 4B are graphs of a comparison of acetaldehyde (FIG. 4A) and hydrogen (FIG. 4B) desorption from the photo-thermal catalysts of the present invention during a fixed photo-irradiation interval as a function of catalyst temperature.

FIGS. 5A and 5B are graphs of the effect of temperature on photoreactions of ethanol to acetaldehyde (FIG. 5A) and hydrogen (FIG. 5B) on 3 wt. % Ag/TiO₂.

FIGS. 6A and 6B are graphs of H₂ (FIG. 6A) and acetaldehyde (FIG. 6B) production on similar amounts of TiO₂ and 3 wt. % Ag/TiO₂ catalyst under thermal and photo-thermal conditions as a function of temperature at ethanol concentration of 3.5 μmole mL⁻¹ in an ethanol:N₂ mixture.

While the invention is susceptible to various modifications and alternative forms, specific embodiments thereof are shown by way of example in the drawings and may herein be described in detail. The drawings may not be to scale.

### DETAILED DESCRIPTION OF THE INVENTION

A solution to the problems (e.g., low rates of hydrogen production due to catalyst deactivation and/or fast electron-hole recombination) associated with currently available methods and/or catalysts to produce hydrogen through photocatalytic dehydrogenation of alcohols has been discovered. The solution is premised on irradiating a thermally-heated photocatalyst having alcohol adsorbed on its surface. The photo-thermal catalyst can be a photo-active metal oxide and can optionally include a plasmon resonance metal dispersed on the photo-active metal oxide. Without wishing to be bound by theory, it is believed that thermally heating the photocatalyst prior to or during irradiation facilitates interaction between the photo-active material and the alcohol adsorbed on the catalyst surface. This results in increased hydrogen and the organic product production when compared with the same catalyst that has not been thermally heated (e.g., temperature of catalyst is room temperature or 15° C. to 30° C.). Furthermore, the present invention offers a commercially viable hydrogen production process from a feed source that can be based on alcohols (e.g., bio-based ethanol) rather than fossil fuels.

These and other non-limiting aspects of the present invention are discussed in further detail in the following sections.

A. Method and System for Producing Hydrogen from an Alcohol

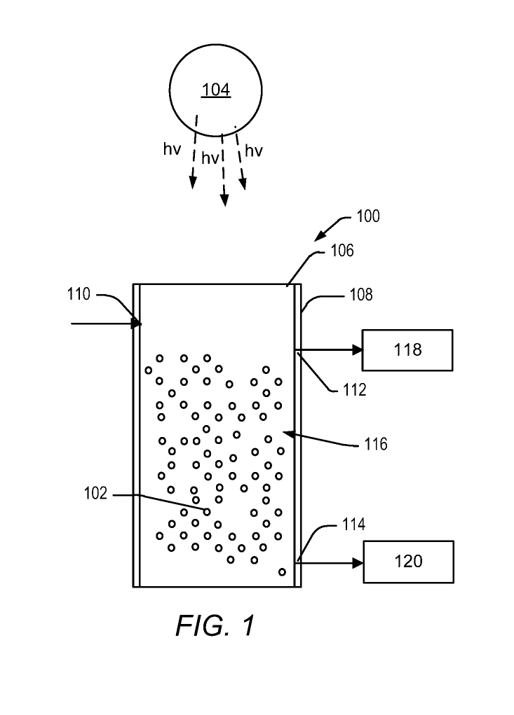

Methods of producing hydrogen and a dehydrogenated alcohol compound (e.g., an aldehyde or ketone) are described throughout the specification and Examples. Referring to FIG. 1, system 100 is illustrated, which can be used to convert alcohols to hydrogen and the corresponding organic product with photo-thermal catalysts 102 of the present invention. System 100 can include light source 104, reactor 106, heating source 108, alcohol feed stream inlet 110, hydrogen outlet 112, and product outlet 114.

Reactor 106 can include reaction zone 116 having the photo-thermal catalyst 102 of the present invention. A non-limiting example of reactor 106 that can be used is a fixed-bed reactor (e.g., a fixed-bed tubular quartz reactor, which can be operated at atmospheric pressure or a flow through quartz reactor). The amount of photo-thermal catalyst 102 used can be modified as desired to achieve a given amount of product produced by system 100.

The alcohol feed stream can be configured to be in fluid communication with reactor 106 via alcohol source inlet 110. Alcohol feed stream inlet 110 can be configured (e.g., valves, controllers and the like) such that the amount of alcohol feed entering reactor 106 can be regulated. The alcohol feed stream (e.g., ethanol, propanol, or methanol) can enter reactor 106 through alcohol feed stream inlet 110 and contact the photo-thermal catalyst 102 for a time sufficient to adsorb the alcohol in the alcohol feed stream on the photocatalyst. The alcohol feed stream can include 90 vol. % to 100 vol. %, or 90 vol. %, 91 vol. %, 92 vol. %, 93 vol. %, 94 vol. %, 95 vol. %, 96 vol. %, 97 vol. %, 98 vol. %, 99 vol. % or 99.9 vol. % alcohol. By way of example, the alcohol source can enter reactor 106 and the pressure in the reaction zone 114 can be reduced (e.g., a vacuum of 1×10⁻⁹ torr) for a period of time sufficient (e.g., 1 min to 60 min, or 1 min to 55 min, 5 min to 40 min, or 10 min to 30 min, or any range or value there between) to adsorb the alcohol onto the photocatalyst. In some embodiments, the photo-thermal catalyst 102 can be heated to remove any water and/or processing solvent, and then contacted with the alcohol under reduced pressure or atmospheric pressure for a time sufficient to adsorb the alcohol on the thermally heated photocatalyst. In some embodiments, the alcohol feed stream can flow into the reactor as a mixture of alcohol and inert gas (e.g., nitrogen or argon gas). In some embodiments, the alcohol can be removed from reactor 106 prior to heating the reactor.

Heating source 108 can be configured to heat photo-thermal catalyst 102 to a temperature sufficient to facilitate conversion of the alcohol absorbed on the surface of the catalyst to hydrogen and an organic product (e.g., aldehydes and ketones). A non-limiting example of heating source 108 can be a temperature controlled heater, a heat exchanger, steam jacketed reactor or the like. Photo-thermal catalyst 102 can be heated to an average temperature of 150° C. to 400° C., 250° C. to 400° C., 300° C. to 400° C., or 325° C. to 375° C., or 150° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 305° C., 310° C., 315° C., 320° C., 325° C., 330° C., 335° C., 340° C., 345° C., 350° C., 355° C., 360° C., 365° C., 370° C., 375° C., 380° C., 385° C., 390° C., 400° C., or any range or value there between during irradiation or prior to irradiation. The temperature can be the surface temperature of the catalyst. In other embodiments, the temperature is the reactor temperature obtained by positioning a thermocouple near the catalyst in a reactor. In some embodiments, photo-thermal catalyst 102 can be heated prior to be placed in reactor 106. For example, photo-thermal catalyst 102 can be heated in an oven and then transferred to reactor 106.

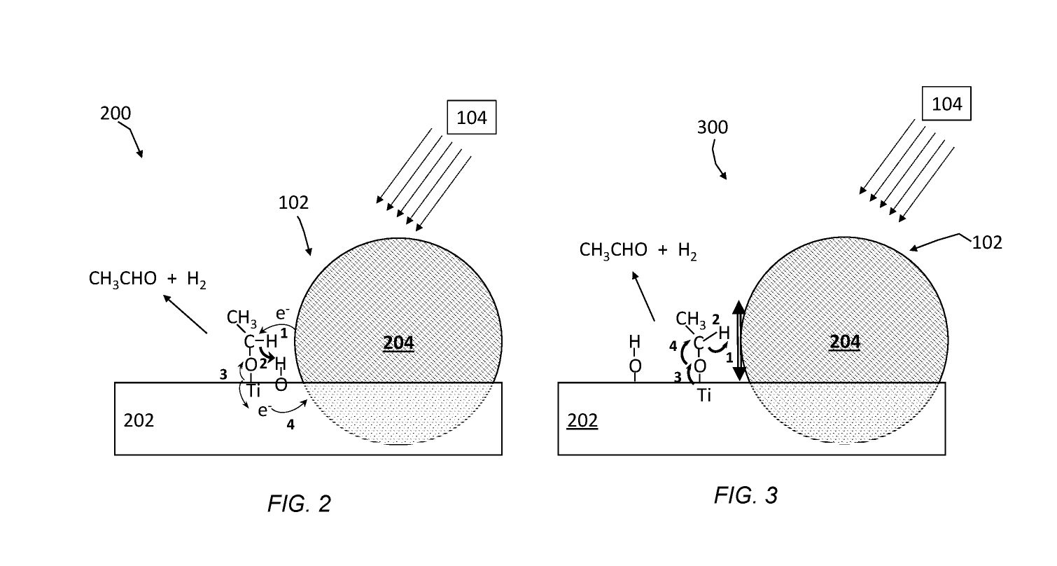

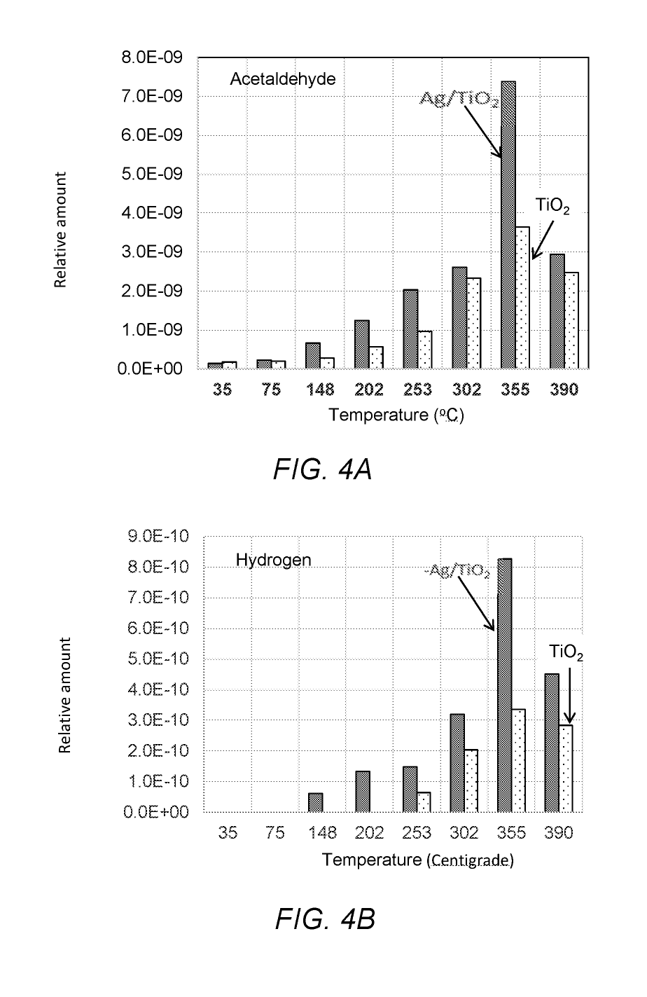

The thermally heated photo-thermal catalyst 102 having alcohol absorbed on the photo-thermal catalyst can be irradiated by applying electromagnetic radiation from light source 104 to the photo-thermal catalyst. The electromagnetic radiation can include ultraviolet radiation, visible light, infrared radiation, or any combination thereof. In some particular instances, the electromagnetic radiation can have a wavelength of 100 nm to 1000 nm, 300 nm to 800 nm, 400 nm to 600 nm, 450 to 550 nm, or 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm or any range or value there between. In some embodiments, irradiation is performed in the presence of an alcohol feed source. By way of example, alcohol feed stream inlet 110 can be closed and the flow of the alcohol feed stream can be discontinued prior to irradiation or the thermally heating the photo-thermal catalyst by closing alcohol feed stream inlet 110. Alternatively, the thermally-heated photo-thermal catalyst can be placed in the alcohol feed stream or subjected to a flow of alcohol feed stream during the irradiation. Inlet 110 and outlets 112 and 114 can be open and closed as desired to maintain the desired concentrations of reactants and products. In some embodiments, photo-thermal catalyst 102 can be heated and irradiated simultaneously after alcohol has been adsorbed on the catalyst. It should be understood that the order of heating and irradiation can be varied, however, the production of the organic products and/or hydrogen can decrease in the absence of irradiation. Without wishing to be bound by theory, it is believed that heating the photo-thermal catalyst during irradiation facilitates the transfer of electrons from the photocatalyst to the alcohol, and thereby increases the rate of hydrogen production from the alcohol. Said another way, electron-hole recombination and by-product formation are diminished and dehydrogenation is increased. It is also believed that once the alcohol has been converted to hydrogen and other products and desorbed from the thermal-photocatalyst, more alcohol can adsorb on the catalyst and/or contact the photo-thermal catalyst to continue the reaction cycle. FIGS. 2 and 3 are illustrations of proposed reaction mechanisms 200 and 300 for the formation of H₂ and acetaldehyde during irradiation of a thermally heated photocatalyst having alcohol absorbed on its surface. As shown in FIGS. 2 and 3, photo-thermal catalyst 102 having alcohol (shown as methanol) adsorbed on the surface of the photoactive metal oxide (MO) substrate 202 can undergo “hot electron transfer” (FIG. 2) and polarization of the adsorbate (FIG. 3), both of which result in formation of H₂ and acetaldehyde. “Hot electrons” are high energy electrons that tunnel out of the photo-active metal oxide substrate. As shown in FIG. 2, irradiation from light source 104 of the thermally heated photo-thermal catalyst excites the photoactive catalyst having a photo-active metal oxide substrate 202 (e.g., TiO₂) and a plasmonic metal 204 (e.g., Ag), which results in a “hot electron transfer” from the metal oxide/plasmonic metal to the C—H bond (arrow 1). Transfer of the hot electron can result in accelerating C—H bond breakage and hydride (H⁻) transfer to H—O in metal oxide substrate 202 (arrow 2), followed by, or simultaneous, hemolytic cleavage of the O-M bond (e.g., O—Ti bond, arrows 3) to form acetaldehyde, and subsequently electron transfer of the electrons (arrow 4) from the hemolytic cleavage back to plasmonic metal 204 to reduce the metal and continue the cycle. As shown in FIG. 3, irradiation of thermally heated photocatalyst 102 can promote polarization of the adsorbate, which can accelerate hydride transfer through elongation of the C—H bond of the alcohol. Irradiation of the thermally heated photocatalyst can establish an electronic field effect between a H of the alcohol (arrow 1) and plasmonic metal 204, which can result in elongation of the C—H “transition state” (ref. no. 2), followed by, or simultaneous, heterolytic cleavage of the O—Ti bond (arrow 3) to form acetaldehyde (i.e., through formation of a double bond between the O and C of the alcohol) (arrow 4). Pathways shown in FIGS. 2 and 3 can occur simultaneously or at different times throughout the catalytic cycle.

Referring back to FIG. 1, hydrogen produced by contact of the alcohol with the photo-thermal catalyst can exit reaction zone 116 through hydrogen outlet 112 and be collected in collection device 118. In some embodiments, hydrogen outlet is membrane selective to hydrogen. Hydrocarbon products (e.g., aldehydes, ketones and by-products) can exit the reaction zone 116 via product outlet 114 and be collected in collection device 120. In preferred aspects, the majority of the products produced are hydrogen and aldehydes and/or ketones (e.g., when isopropanol is used as an alcohol source). However, the product stream can include by-products (e.g., olefins or crontaldehyde, and unreacted alcohol). Collection devices 118 and 120 can be in fluid communication with reactor 106 via the outlets 110 and 112, respectively. Collection devices 118 and 120 can be configured to store, further process, or transfer the desired reaction products (e.g., hydrogen and/or aldehydes or ketones) for other uses.

### B. Photo-Thermal Catalysts

The photo-thermal catalysts of the present invention can include a photo-active metal oxide and, optionally, a plasmon resonance material. The photo-thermal catalyst has a temperature of 150° C. to 400° C. and is in contact with electromagnetic radiation.

The photo-active metal oxide can include any metal oxide able to be excited by light in a range from 100-500 nanometers (e.g., a n-type semiconductor material). Non-limiting examples of the metal oxide are titanium dioxide (TiO₂), cerium dioxide (CeO₂), zinc oxide (ZnO), or vanadium oxide (V₂O₅), or any combination thereof. The metal oxide can be obtained from commercial sources (e.g., Sigma-Aldrich® (USA), or made as described below. Titanium dioxide can be in the form of three phases, the anatase phase, the rutile phase, and the brookite phase. Anatase and rutile phases have a tetragonal crystal system, whereas the brookite phase has an orthorhombic crystal system. While anatase and rutile both have a tetragonal crystal system consisting of TiO₆ octahedra, their phases differ in that anatase octahedras are arranged such that four edges of the octahedras are shared, while in rutile, two edges of the octahedras are shared. These different crystal structures resulting in different density of states may account for the different efficiencies observed for transfer of charge carriers (electrons) in the rutile and anatase phases and the different physical properties of the catalyst. For example, anatase is more efficient than rutile in the charge transfer, but is not as durable as rutile. Each of the different phases can be purchased from various manufactures and supplies (e.g., titanium (IV) oxide anatase nanopowder and titanium (IV) oxide rutile nanopowder in a variety of sizes and shapes can be obtained from Sigma-Aldrich® Co. LLC (St. Louis, Mo., USA) and from Alfa Aesar GmbH & Co KG, A Johnson Matthey Company (Germany)) and all phases of titanium dioxide from L.E.B. Enterprises, Inc. (Hollywood, Fla. USA)). TiO₂ phases can also be synthesized using known sol-gel methods (See, for example, Chen et al., Chem. Rev. 2010 Vol. 110, pp. 6503-6570, the contents of which are incorporated herein by reference). In one aspect of the invention, mixed phase titanium dioxide anatase and rutile may be the transformation product obtained from heat-treating single-phase titanium dioxide anatase at selected temperatures. Heat-treating the single-phase titanium dioxide anatase nanoparticle produces small particles of rutile on top of anatase particles, thus maximizing the interface between both phases and at the same time allowing for a large number of adsorbates (water and ethanol) to be in contact with both phases, due to the initial small particle size. Single-phase TiO₂ anatase nanoparticles that are transformed into mixed phase TiO₂ nanoparticles have a surface area of about 45 to 80 m²/g, or 50 m²/g to 70 m²/g, or preferably about 50 m²/g. The particle size of these single-phase TiO₂ anatase nanoparticles is less than 95 nanometers, less than 50 nm, less than 20, or preferably between 10 and 25 nm. Heat treating conditions can be varied based on the TiO₂ anatase particle size and/or method of heating (See, for example, Hanaor et al. in Review of the anatase to rutile phase transformation, J. Material Science, 2011, Vol. 46, pp. 855-874), and are sufficient to transform single-phase titanium dioxide to mixed phase titanium dioxide anatase and rutile. Other methods of making mixed phase titanium dioxide materials include flame pyrolysis of TiCl₄, solvothermal/hydrothermal methods, chemical vapor deposition, and physical vapor deposition methods. A non-limiting example of transforming nanoparticles of TiO₂ anatase nanoparticles to mixed phase TiO₂ anatase and rutile nanoparticles includes heating single-phase TiO₂ anatase nanoparticles isochronally at a temperature of 700-800° C. for about 1 hour to transform the nanoparticles of TiO₂ anatase phase to nanoparticles of mixed phase TiO₂ anatase phase and rutile phase. In a preferred embodiment, titanium dioxide anatase can be heated to a temperature of 780° C. to obtain mixed phase titanium dioxide containing about 37% rutile. Without wishing to be bound by theory, it is believed that this ratio and the particle structure may allow for the efficient transfer of charge carriers (electrons) from the rutile phase to the anatase phase, where said charge carries in the anatase phase have an increased chance of being transferred to the metal conducting materials rather than undergoing an electron-hole recombination event. The percentage of anatase to rutile in the titanium dioxide polymorph can be determined using powder X-ray diffraction (XRD) techniques. For example, a Philips X'pert-MPD X-ray powder diffractometer may be used to analyze powder samples of titanium dioxide polymorphs. Using the areas of these peaks the amounts of rutile phase in the titanium dioxide polymorph can be determined using the following equation:

$$\% rutile = \frac{1}{( \frac{A}{R} \times 0.884 ) + 1} \times 100 \qquad ( 1 )$$

where A is the area of anatase peak (such as that of the (101) plane); R is the area of rutile peak (such as that of the (101) plane); as determined by XRD; and 0.884 is a scattering coefficient.

Notably, it was discovered that when a ratio of anatase to rutile of 1.5:1 or greater is used, the photocatalytic activity of the titanium dioxide can be substantially increased. The mixed phase TiO₂ nanoparticles of the present invention can have a ratio of anatase and rutile phase ranges from 1.5:1 to 10:1, from 6:1 to 5:1, from 5:1 to 4:1, or from 2:1, 3:1, 4:1, 5:1, 6:1, 7:1, 8:1, 9:1, 10:1 or any range or value there between. As explained above, it is believed that this ratio allows for the efficient transfer of charge carriers (electrons) from the rutile phase to the anatase phase, where said charge carries in the anatase phase have an increased chance of being transferred to the metal conducting materials rather than undergoing an electron-hole recombination event.

The photo-thermal catalyst can include a photo-active metal oxide and a plasmon resonance material. The plasmon resonance material can be metal or metal alloys. The metal or metal alloys can be obtained from a variety of commercial sources in a variety of forms (e.g., particles, phases, rods, films, etc.) and sizes (e.g., nanoscale). Non-limiting examples of commercial sources for plasmon resonance material include Sigma-Aldrich® Co. LLC and Alfa Aesar GmbH & Co KG offer. Alternatively, they can be made by any process known by those of ordinary skill in the art. In a non-limiting aspect, the metal particles can be prepared using co-precipitation or deposition-precipitation methods. The metal particles can be used as conductive material for the excited electrons to ultimately reduce hydrogen ions to produce hydrogen gas. The metal particles can be substantially pure particles of Au, Cu, and Ag. The metal particles can also be binary or tertiary alloys of Au, Cu, and/or Ag. The metal particles are highly conductive materials, making them well suited to act in combination with the photoactive material to facilitate transfer of excited electrons to hydrogen before an electron-hole recombination event occurs or by increasing the time that such an event occurs. The metal particles can also enhance efficiency via resonance plasmonic excitation from visible light, enabling capture of a broader range of light energy. The metal particles can be of any size compatible with the metal oxide. In some embodiments, the metal particles are nanostructures. The nanostructures can be of any form suitable for use in the photoactive catalytic systems of the present invention. Non-limiting examples of nanostructure forms include nanowires, nanoparticles, nanoclusters, nanocrystals, or combinations thereof. In a preferred aspect, the metals have an average particle size of 30 nm or less, or 1 to 30 nm, 5 to 10 nm, or 6 to 7 nm, or 30 nm, 29 nm 28 nm, 27 nm, 26 nm, 25 nm, 24 nm, 23 nm, 22 nm, 21 nm, 20 nm, 19 nm, 18 nm, 17 nm, 16 nm, 15 nm, 14 nm, 13 nm, 11 nm, 10 nm, 9 nm, 8 nm, 7 nm, 6 nm, 5 nm, 4 nm, 3 nm, 1 nm or less, or any range or value there between as determined by transmission electron microscopy (TEM). The photo-thermal catalyst can include 0.1 to 10 wt. %, 0.3 to 5 wt. % or 0.5 to 3 wt. %, or 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 1.25 wt. % 1.50 wt. %, 1.75 wt. %, 2 wt. %, 2.25 wt. %, 2.5 wt. %, 2.75 wt. %, 3.0 wt. %, 3.5 wt. %, 4.0 wt. %, 4.5 wt. %, 5 wt. %, 6 wt. %, 7 wt. %, 8 wt. %, 9 wt. %, 10 wt. % or any range or value there between of the plasmon resonance active metal, based on the total weight of the photo-thermal catalyst.

The plasmon resonance metal doped photo-thermal catalyst can be made using known catalyst preparation methods. A non-limiting example of a method that can be used to make photoactive catalysts 102 of the present invention includes formation of an aqueous solutions of metal oxide particles and plasmonic resonance metals, (for example, Au, Ag, and Cu precursors), followed by precipitation, where the metal particles are attached to at least a portion of the surface of the precipitated photo-active metal oxide. Alternatively, the metal particles can be deposed on the surface of the photo-active metal oxide by any process known by those of ordinary skill in the art. Deposition can include attachment, dispersion, and/or distribution of the metal particles on the surface of the photo-active active metal oxide.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Production of Hydrogen and Acetaldehyde from Ethanol

All experiments were performed in an ultra-high vacuum chamber (base pressure 1.0×10⁻⁹ torr) equipped with sputter gun to clean the sample and a mass spectrometer to monitor gas phase products produced under photo-thermal conditions. A known amount of each of Ag/TiO₂ (29.8 g) or TiO₂ (21.7 g) catalyst was pressed in a circular pellet (1.0 cm diameter) using a hydraulic press (6-7 ton). The pellet was loaded into a jacket made up of Tantalum foil (0.2 mm thick) inside the reactor. A K-type thermocouple was attached to a TiO₂ (110) single crystal piece (2×2 mm) attached to the tantalum jacket at a small distance (≤1 mm) from the pellet to monitor the temperature. Each experiment was started by sample annealing to 500° C. to remove any adsorbed ethanol or water followed by its cooling to 35° C. (30-60 min). Ethanol was dosed (64 L; 1 L=10⁻⁶ torr s) by increasing the background pressure to 1×10⁻⁶ torr by leaking clean ethanol for 64 seconds using a leak valve. The sample was put in front of the mass spectrometer. Prior to experiments, the temperature of the sample pallet was increased to 350° C. to make sure that the same ethanol coverage was obtained before each photoreaction experiment. Then the sample temperature was adjusted to the desired temperatures of 35° C., 75° C., 204° C., 253° C., 302° C., and 355° C., and mass spectrometer signal for each fragment measured was allowed to stabilize (30-60 min). The sample was irritated with UV radiations from a 300 W Xenon lamp operated at 100% intensity using a fiber optic waveguide through a fused silica window. A 360 nm short-pass filter was used to allow UV radiation with wavelength range of 310-410 nm. The UV radiations from a 300 W Asahi Compact Xenon lamp Max 303 with 410 nm pass filter was used, however, this filter is not necessary. Radiation were guided to the sample using fiber optics and convex lenses. The flux on the sample was measured approximately 11.2 mW/cm². The flux was measured using Spectral Evolution Spectro-radiometer model SR500 (operation range 320 nm to 1100 nm).

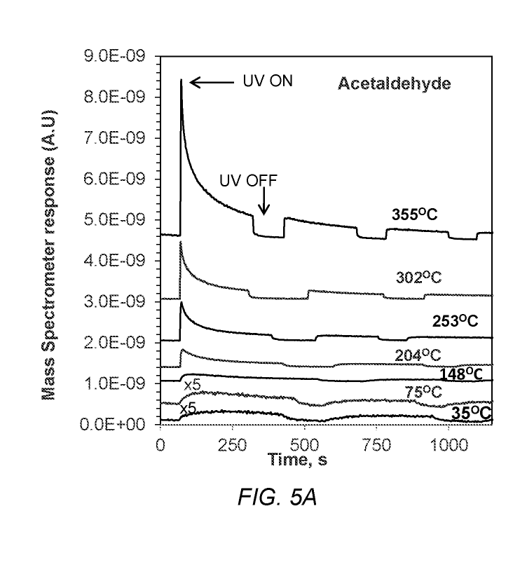

FIGS. 4A and 4B depict a comparison of acetaldehyde (FIG. 4A) and hydrogen (FIG. 4B) desorption during a fixed photo-irradiation interval as a function of catalyst temperature. The amount produced were normalized to catalyst weight. The amount of acetaldehyde and hydrogen presented in FIGS. 4A and 4B are directly proportional to their rate of formation as they are obtained during a constant photo-irradiation interval at different temperature as indicated. As shown, acetaldehyde and hydrogen production rates were enhanced on neat and Ag supported TiO₂ by increasing the temperature of the photocatalyst using the photo-thermal reaction conditions of the present invention. Without wishing to be bound by theory, it is believed that the higher production rates for Ag supported TiO₂ was related to surface plasma resonance mechanism. As shown in FIGS. 4A and 4B, a three-fold increase in acetaldehyde (approx. ×1000) where a one fold (approx. ×10) increase in hydrogen production rates was observed for the Ag/TiO₂ catalyst. The decrease in photoreaction product amounts at 390° C. was attributed to lower ethanol coverage on the photocatalyst as adsorbed ethanol starts to desorb at a temperature of 105° C. until it starts converting into other products above 325° C. Less than 10% of initial ethanol coverage was left at temperatures above 375° C. The photo-thermal method of the present reaction can deliver hydrogen production rates high enough for industrial application.

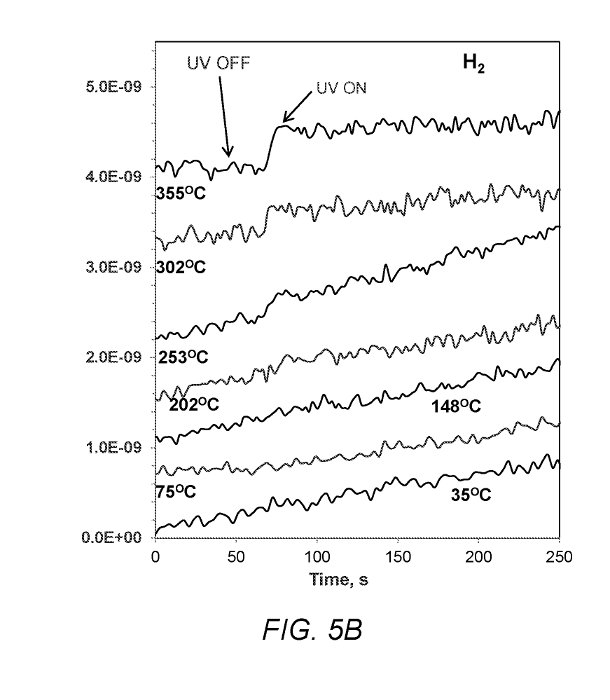

FIGS. 5A and 5B depict photoreaction results for the production of hydrogen and ethanol using the photo-thermal catalyst of the present invention. It was observed that the rate of acetaldehyde production on UV light exposure increased with an increase in temperature indicated by the height of the spike in FIGS. 5A and 5B. The amount of acetaldehyde and hydrogen produced was monitored using mass spectrometry (m/z=29 and m/z=2 mass fragments). The intensity of m/z=29 mass fragment signal shown (FIG. 5A) was obtained by subtracting the contribution from ethanol, while the intensity of m/z=2 signal (FIG. 5B) was obtained after subtracting the contribution from acetaldehyde and water. Initially the increase in hydrogen production rate was not very clear due to the high m/z=2 background, however, a clear increase in signal was seen at high temperature due to large amount of hydrogen produced.

### Example 2

### Production of Hydrogen and Acetaldehyde at Steady State Conditions

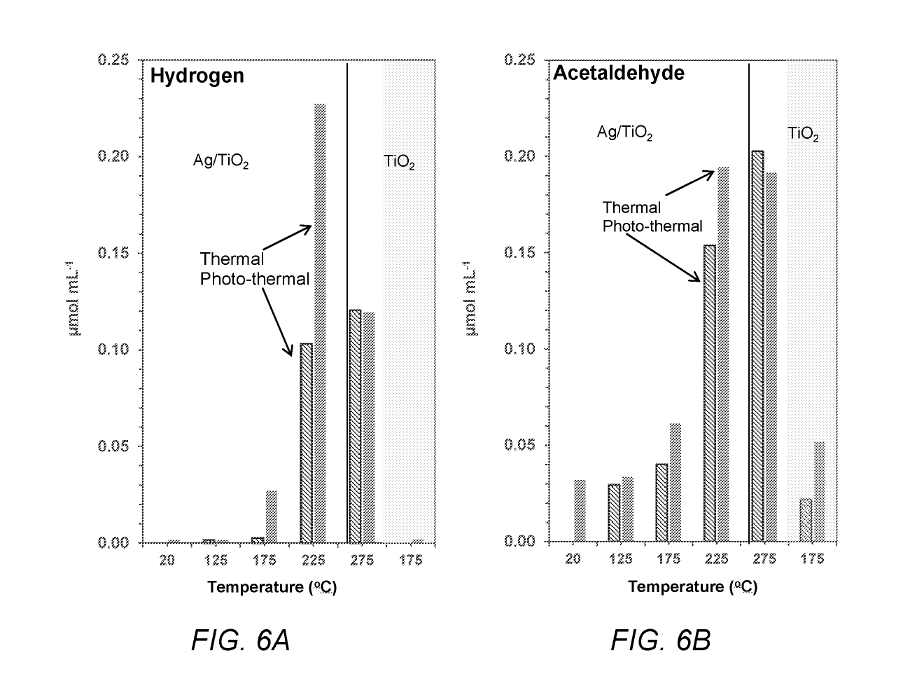

TiO₂, as a control) or Ag/TiO₂ catalyst (31 mg, 3 wt. % Ag based on the total weight of the catlayst) was coated on a Pyrex glass slide (0.9 cm×6.2 cm) and placed in a horizontal Pyrex reactor (diameter: outer=1.3 cm; inner=1.1 cm). A type K thermocouple was placed close to the catalyst slide inside the reactor. The reactor was heated to the desired temperature (from 20 to 275° C.) and ethanol flow was maintained by bubbling N₂ gas (99.99999% purity) through an ethanol (Sigma-Aldrich, anhydrous ≥99.5%) reservoir. Before each experiment, the catalyst was subjected to oxidation to remove accumulation of carbon followed by reduction using high purity (99.9999% purity) O₂ and H₂ gas. Both treatments were carried at 250° C. for one hour each at gas flow rate of 20 mL min⁻¹. The removal of carbon and oxidation of catalysts was confirmed by the appearance of white catalyst color. The catalyst reduction was confirmed by the appearance of black color due to the reduction of silver oxide to silver metal. A 100 Watt ultraviolet lamp (H-144GC-100, Sylvania par 38) was used as a UV source with a flux of 4 mWatt cm⁻² with the cut off filter (360 nm and above). Gas samples of 0.5 ml were taken from the flow stream after the catalyst slide using a gas tight syringe and analyzed using gas chromatograph. The gas chromatograph was equipped with HayeSep-Q packed column (36×0.125 inches) internally coated with 2 mm thick polydivinylbenzene layer. The initial column temperature was set at 40° C. for 3 min. to analyze H₂ and other low molecular weight gases then ramped to 200° C. at 30° C. mid′. N₂ gas was used as carrier gas at flow rate of 20 mLmin⁻¹ and at eight psi inlet pressure. The H₂, ethanol, acetaldehyde, acetone, ethylene, ethane, methane and CO₂ were monitored. FIGS. 6A and 6B shows graphs of H₂ (FIG. 6A) and acetaldehyde (FIG. 6B) production on similar amounts (31 mg) of TiO₂ and 3 wt. % Ag/TiO₂ catalyst under thermal and photo-thermal conditions as a function of temperature at ethanol concentration of 3.5 μmole mL⁻¹ in an ethanol:N₂ mixture. Table 1 lists H₂ and acetaldehyde production on similar amounts (31 mg) of TiO₂ and 3 wt. % Ag/TiO₂ catalyst under thermal (T) and photo-thermal (PT) conditions as a function of temperature at ethanol concentration of 3.5 μmole mL⁻¹ in the ethanol:N₂ mixture, under steady state reactions conditions.

TABLE 1

| | H₂ production | | | | | | | |
|---|---|---|---|---|---|---|---|---|
| | (μmole mL⁻¹) | | | Acetaldehyde production | | | | |
| Temp. | | | PT/T | (μmole mL⁻¹) | | | Acetadehyde:H₂ ratio | |
| (° C.) | T | PT | ratio | T | PT | PT/T | T | PT |
| 20 | — | 1.5 × 10⁻³ | — | — | 3.2 × 10⁻² | — | — | 21.3 |
| 125 | 1.6 × 10⁻³ | 1.5 × 10⁻³ | 1 | 3.0 × 10⁻² | 3.3 × 10⁻² | 1 | 18.7 | 22.0 |
| 175 | 2.7 × 10⁻³ | 2.7 × 10⁻² | 10 | 4.0 × 10⁻² | 6.1 × 10⁻² | 1.5 | 14.8 | 2.2 |
| | (—) | (2.0 × 10⁻³) | | (2.2 × 10⁻²) | (5.2 × 10⁻²) | (2.4) | | |
| 225 | 1.0 × 10⁻¹ | 2.3 × 10⁻¹ | 2.3 | 1.5 × 10⁻¹ | 1.9 × 10⁻¹ | 1.3 | 1.5 | 0.8 |
| 275 | 1.2 × 10⁻¹ | 1.2 × 10⁻¹ | 1 | 2.0 × 10⁻¹ | 1.9 × 10⁻¹ | 1 | 1.7 | 1.6 |

T = Thermal,
PT = Photothermal

FIGS. 6A and 6B, and Table 1 show the concentrations and relative concentrations of hydrogen and acetaldehyde produced as a function of temperature under thermal (dark) and photo-thermal (light) conditions. From the data, it was determined that no synergism occurred at 125° C. as similar amounts of both H₂ and acetaldehyde were produced under thermal and photo-thermal reaction conditions. A 10 and 2.3 times increase in H₂ while 1.5 and 1.3 times increase in acetaldehyde production in the case of photo-thermal when compared to thermal reactions was observed at 175° C. and 225° C., respectively. At 275° C., both products have the same rate. Ethane, ethylene and acetone were also observed at this temperature, indicating the occurrence of other reactions apart from ethanol dehydrogenation reaction. From columns 8 and 9 of Table 1, a large proportion of H₂ produced was converted to H₂O with the inevitable background O₂. In spite of a similar enhancement in acetaldehyde production for the photo-thermal reactions at 175° C. (when compared to thermal reactions), a 14-times higher H₂ production in the case of Ag/TiO₂, when compared to TiO₂, was observed. Without wishing to be bound by theory, it is believed that the light contributed to hydrogen production by generating excited electrons (that reduced hydrogen ions of surface hydroxyls). The presence of a metal (Ag) was needed for H ion reduction by excited electrons from the conduction band of TiO₂ as well as the consequent hydrogen atoms association to produce molecular H₂.

1. A photo-thermal method for producing hydrogen (H₂) and an organic product from alcohol, the method comprising irradiating a thermally-heated metal oxide photocatalyst that includes alcohol adsorbed on the surface of the photocatalyst with electromagnetic radiation to produce H₂ and the organic product from the alcohol, wherein the thermally-heated metal oxide photocatalyst has a temperature of 150° C. to 400° C.

2. The photo-thermal method of claim 1, wherein the alcohol is C₁₋₃ alcohol and hydrogen and the organic product are formed by dehydrogenation of the alcohol.

3. The photo-thermal method of claim 1, wherein the thermally-heated metal oxide photocatalyst has a temperature of 250° C. to 400° C.

4. The photo-thermal method of claim 1, wherein the metal oxide photocatalyst comprises titanium dioxide (TiO₂), cerium dioxide (CeO₂), zinc oxide (ZnO), or vanadium oxide (V₂O₅) or any combination thereof.

5. The photo-thermal method of claim 4, wherein the metal oxide is titanium dioxide (TiO₂).

6. The photo-thermal method of claim 1, wherein the metal oxide is cerium dioxide (CeO₂).

7. The photo-thermal method of claim 1, wherein the metal oxide photocatalyst comprises a plasmon resonance active metal dispersed on the thermally-heated metal oxide photocatalyst.

8. The photo-thermal method of claim 7, wherein the plasmon resonance active metal is silver (Ag), gold (Au), Copper (Cu), or any combinations thereof or alloys thereof.

9. The photo-thermal method of claim 7, wherein the thermally-heated metal oxide photocatalyst comprises 0.1 to 10 wt. % or 0.3 to 5 wt. % or 0.5 to 3 wt. % of the plasmon resonance active metal.

10. The photo-thermal method of claim 1, wherein the thermally-heated metal photocatalyst is subjected to an alcohol feed stream to adsorb the alcohol prior to the irradiation, or the thermally-heated metal oxide photocatalyst is subjected to an alcohol feed stream during the irradiation.

11. The photo-thermal method of claim 1, wherein the electromagnetic radiation has a wavelength of 100 nm to 1000 nm, preferably 300 nm to 500 nm.

12. The photo-thermal method of claim 1, wherein the electromagnetic radiation comprises of ultraviolet radiation or sunlight.

13. The photo-thermal method of claim 1, wherein the production of the aldehyde decreases in the absence of irradiation.

14. A photo-thermal catalyst comprising a photo-active metal oxide and alcohol adsorbed on the surface of the catalyst, wherein the catalyst has a temperature of 150° C. to 400° C. and is in contact with electromagnetic radiation.

15. The photo-thermal catalyst of claim 14, wherein the alcohol is a C₁₋₃ alcohol, and the catalyst is capable of producing hydrogen and an organic product from dehydrogenation of the alcohol.

16. The photo-thermal catalyst of claim 14, wherein the catalyst has a temperature of 250° C. to 400° C.

17. The photo-thermal catalyst of claim 14, wherein the photo-active metal oxide comprises a plasmon resonance active metal dispersed on the thermally-heated metal oxide.

18. The photo-thermal catalyst of claim 17, wherein the plasmon resonance active metal is silver (Ag), gold (Au), Copper (Cu), or any combinations or oxides or alloys thereof.

19. The photo-thermal catalyst of claim 19, wherein the metal oxide comprises titanium dioxide (TiO₂), cerium dioxide (CeO₂), zinc oxide (ZnO), or vanadium oxide (V₂O₅) or any combination thereof.

20. The photo-thermal catalyst of claim 18, wherein the photocatalyst is Ag/TiO₂.
